(19) United States
(12) Patent Application Publication
(10) Pub. No.: US 2026/0248405 A1
(43) Pub. Date: Aug. 27, 2026

(54) BIOIMPEDANCE RING SENSOR FOR PHYSIOLOGICAL MONITORING

(71) Applicant: The Texas A&M University System, College Station, TX (US)

(72) Inventors: Roozbeh Jafari, Austin, TX (US); Kaan Sel, College Station, TX (US); Deen Osman, Bryan, TX (US)

(73) Assignee: The Texas A&M University System, College Station, TX (US)

(21) Appl. No.: 18/871,061

(22) PCT Filed: Jun. 28, 2023
(86) PCT No.: PCT/US2023/069300

§ 371 Date: Dec. 2, 2024

Related U.S. Application Data
(60) Provisional application No. 63/356,052, filed on Jun. 28, 2022.

(51) Int. Cl. A61B 5/053; A61B 5/00; A61B 5/01; A61B 5/11

(52) U.S. Cl. CPC A61B 5/053; A61B 5/0053; A61B 5/01; A61B 5/1126; A61B 5/6826; A61B 2560/0242; A61B 2560/0468; A61B 2562/0271

(57) ABSTRACT

A bioimpedance ring sensor configured to be worn on a finger of a human subject. The bioimpedance ring sensor may include a ring-shaped body having a central axis, a radially inner annular surface, and a radially outer annular surface. The bioimpedance ring sensor may also include a plurality of electrodes positioned on the radially inner annular surface. The plurality of electrodes may include injection electrodes and sensing electrodes. The bioimpedance ring sensor may also comprise a controller coupled to the plurality of electrodes. The controller may be configured to direct an electric current to at least one of the injection electrodes, to detect a voltage potential via at least one of the of sensing electrodes, and to determine bioimpedance associated with the human subject based upon the electric current and the voltage potential.

### CROSS REFERENCE TO RELATED APPLICATIONS

This application is a 35 U.S.C. § 371 national stage application of PCT/US2023/069300 filed Jun. 28, 2023, and entitled “Bioimpedance Ring Sensor for Physiological Monitoring,” which claims priority to U.S. Provisional Application Ser. No. 63/356,052 filed Jun. 28, 2022 and entitled “Bioimpedance Ring Sensor for Physiological Monitoring,” each of which is incorporated herein by reference in its entirety for all purposes.

### TECHNICAL FIELD

The present disclosure generally relates to a bioimpedance ring sensor for physiological monitoring. More particularly, the present disclosure relates to bioimpedance sensor configured to be worn on and/or around a finger of a user or wearer so as to monitor various physiological parameters.

### BACKGROUND

Cardiovascular disease (CVD) has become the leading cause of death in various countries, accounting for approximately one-third of all deaths globally. The American Heart Association suggests that the direct and total cost of CVD in the U.S. is projected to exceed $750 billion and $1.1 trillion in 2035. Complex hemodynamic parameters, such as blood pressure (BP) are indicators for determining proper cardiovascular system function among other health-related metrics.

Electrical impedance (or more simply “impedance”) can be used to detect or measure a number of properties within biological systems (e.g., a human or animal body). Impedance of such a biological system may be referred to as “bioimpedance” or “Bio-Z.” In some circumstances, bioimpedance may be utilized to measure or detect various properties associated with a circulatory system (or portion thereof) due to the differences in impedance of tissue and fluids (e.g., blood). For instance, bioimpedance measurements may be used to determine various attributes or parameters of the circulatory system and/or the biological system more broadly, such as, for example: blood pressure, vasoconstriction, vasodilation, arterial stiffness and/or compliance, body composition, muscle activity, electrodermal activity, skin and/or body temperature.

Conventional wearable sensor technologies, such as those using optical photoplethysmography (PPG), are unsuitable for widespread use because these technologies exhibit inaccuracies across differing skin tones and different body fat compositions. In addition, optical sensing is prone to motion artifacts and also requires more processing power, due to the necessity to omit light. Some conventional wearable sensor are limited by their size and inconvenience. For example, brachial blood pressure cuffs are bulky and, as such, are limited as to the times and durations for which they can be used. As such, many conventional wearable sensors are unsuitable for continuous use, for example, continuous blood pressure monitoring, especially during sleep, which is an important predictor of worsening cardiovascular health.

As such, there is a need for improved wearable sensors capable of providing continuous health monitoring with good reliability, accuracy, and usability across various populations.

### SUMMARY

Disclosed herein is a bioimpedance ring sensor configured to be worn on a finger of a human subject. The bioimpedance ring sensor may comprise a ring-shaped body having a central axis, a radially inner annular surface, and a radially outer annular surface. The bioimpedance ring sensor may also comprise a plurality of electrodes positioned on the radially inner annular surface. The plurality of electrodes may comprise a plurality of injection electrodes and a plurality of sensing electrodes. The bioimpedance ring sensor may also comprise a controller coupled to the plurality of electrodes. The controller may be configured to direct an electric current to at least one of the plurality of injection electrodes, to detect a voltage potential via at least one of the plurality of sensing electrodes, and to determine bioimpedance associated with the human subject based upon the electric current and the voltage potential.

Also disclosed herein is a system comprising a first bioimpedance ring sensor configured to be worn on a finger of a human subject. The first bioimpedance ring sensor may comprise a first ring-shaped body having a first central axis, a first radially inner annular surface, and a first radially outer annular surface. The first bioimpedance ring sensor may also comprise a first plurality of electrodes positioned on the first radially inner annular surface. The first plurality of electrodes may comprise a first plurality of injection electrodes and a first plurality sensing electrodes. The first bioimpedance ring sensor may also comprise a first controller coupled to the first plurality of electrodes. The first controller may be configured to direct an electric current to at least one of the first plurality of injection electrodes, to detect a voltage potential via at least one of the first plurality of sensing electrodes, and to determine bioimpedance associated with the human subject based upon the electric current and the voltage potential.

The system may further comprise a second bioimpedance ring sensor configured to be worn on a finger of a human subject. The second bioimpedance ring sensor may comprise a second ring-shaped body having a second central axis, a second radially inner annular surface, and a second radially outer annular surface. The second bioimpedance ring sensor may comprise a second plurality of electrodes positioned on the second radially inner annular surface. The second plurality of electrodes may comprise a second plurality of injection electrodes and a second plurality sensing electrodes. The second bioimpedance ring sensor may comprise a second controller coupled to the first plurality of electrodes. The second controller may be configured to direct an electric current to at least one of the second plurality of injection electrodes, to detect a voltage potential via at least one of the first plurality of sensing electrodes, and to determine bioimpedance associated with the human subject based upon the electric current and the voltage potential.

Also disclosed herein is a method of monitoring a physiological parameter of a human subject. The method may comprise positioning a bioimpedance ring sensor on a finger of the human subject. The bioimpedance ring sensor may comprise a ring-shaped body having a central axis, a radially inner annular surface, and a radially outer annular surface. The bioimpedance ring sensor may also comprise a plurality of electrodes positioned on the radially inner annular surface. The plurality of electrodes may comprise a plurality of injection electrodes and a plurality of sensing electrodes. The bioimpedance ring sensor may also comprise a controller coupled to the plurality of electrodes. The method may also comprise directing an electric current to at least one of the plurality of injection electrodes. The method may also comprise detecting a voltage potential via at least one of the plurality of sensing electrodes. The method may also comprise determining bioimpedance associated with the human subject based upon the electric current and the voltage potential.

### BRIEF DESCRIPTION OF THE DRAWINGS

FIG. 1 depicts an embodiment of a bioimpedance ring sensor according to this disclosure;

FIG. 2 depicts an embodiment of a bioimpedance ring sensor according to this disclosure;

FIG. 3 depicts an embodiment of a bioimpedance ring sensor according to this disclosure:

FIG. 4 depicts an embodiment of a bioimpedance ring sensor according to this disclosure;

FIG. 5 depicts an embodiment of a bioimpedance ring sensor according to this disclosure;

FIG. 6 depicts an embodiment of a bioimpedance ring sensor according to this disclosure;

FIG. 7 depicts an embodiment of a bioimpedance ring sensor according to this disclosure;

FIG. 8 depicts an embodiment of a bioimpedance ring sensor according to this disclosure;

FIG. 9 depicts an embodiment of a bioimpedance ring sensor according to this disclosure;

FIG. 10 depicts an embodiment of a bioimpedance ring sensor according to this disclosure:

FIG. 11 depicts an embodiment of a bioimpedance ring sensor according to this disclosure;

FIG. 12 depicts an embodiment of a bioimpedance ring sensor according to this disclosure;

FIG. 13 depicts an embodiment of a bioimpedance ring sensor according to this disclosure;

FIG. 14 depicts an embodiment of a bioimpedance ring sensor according to this disclosure;

FIG. 15 depicts an embodiment of a bioimpedance sensing system comprising a bioimpedance ring sensor according to this disclosure;

FIG. 16 depicts an embodiment of a bioimpedance sensing system comprising a bioimpedance ring sensor according to this disclosure;

FIG. 17 depicts an embodiment of the implementation of a bioimpedance sensing system comprising a bioimpedance ring sensor according to this disclosure;

FIG. 18 depicts an embodiment of a bioimpedance sensing system comprising a bioimpedance ring sensor according to this disclosure;

FIG. 19 depicts an embodiment of a bioimpedance sensing system comprising a bioimpedance ring sensor according to this disclosure;

FIG. 20 depicts an embodiment of a bioimpedance sensing system comprising a bioimpedance ring sensor according to this disclosure;

FIG. 21 depicts an embodiment of a bioimpedance sensing system comprising a bioimpedance ring sensor according to this disclosure;

FIG. 22 depicts an embodiment of a bioimpedance sensing system comprising a bioimpedance ring sensor according to this disclosure;

FIG. 23 depicts an embodiment of a bioimpedance sensing system comprising a bioimpedance ring sensor according to this disclosure;

FIG. 24 depicts an embodiment of the implementation of a bioimpedance sensing system comprising a bioimpedance ring sensor according to this disclosure;

FIG. 25 depicts certain aspects of the implementation of a bioimpedance ring sensor according to this disclosure;

FIG. 26 depicts certain aspects of the implementation of a bioimpedance ring sensor according to this disclosure; and

FIG. 27 depicts certain aspects of the implementation of a bioimpedance ring sensor according to this disclosure.

### DETAILED DESCRIPTION

To define more clearly the terms used herein, the following definitions are provided. To the extent that any definition or usage provided by any document incorporated herein by reference conflicts with the definition or usage provided herein, the definition or usage provided herein controls.

Regarding claim transitional terms or phrases, the transitional term “comprising”, which is synonymous with “including,” “containing,” “having,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the subject matter described herein. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter the composition or method to which the term is applied.

Within this specification, use of “comprising” or an equivalent expression contemplates the use of the phrase “consisting essentially of,” “consists essentially of,” or equivalent expressions as alternative aspects to the open-ended expression. Additionally, use of “comprising” or an equivalent expression or use of “consisting essentially of” in the specification contemplates the use of the phrase “consisting of,” “consists of,” or equivalent expressions as an alternative to the open-ended expression or middle ground expression, respectively. For example, “comprising” should be understood to include “consisting essentially of,” and “consisting of” as alternative aspects for the aspect, features, and/or elements presented in the specification unless specifically indicated otherwise. The terms “a,” “an,” and “the” are intended, unless specifically indicated otherwise, to include plural alternatives, e.g., at least one. Use of the term “optionally” with respect to any element of a claim is intended to mean that the subject element is required, or alternatively, is not required. Both alternatives are intended to be within the scope of the claim

Features within this disclosure that are provided as minimum values can be alternatively stated as “at least” or “greater than or equal to” any recited minimum value for the feature disclosed herein. Features within this disclosure that are provided as maximum values can be alternatively stated as “less than or equal to” for the feature disclosed herein.

Disclosed herein are various embodiments of a bioimpedance ring sensor configured for monitoring various physiological parameters. As used herein, the term “ring sensor” refers to a sensor, for example, a bioimpedance sensor, that is configured to be worn on the finger of a human subject. The ring sensor disclosed herein may generally include a ring-shaped body, a plurality of electrodes, and a controller.

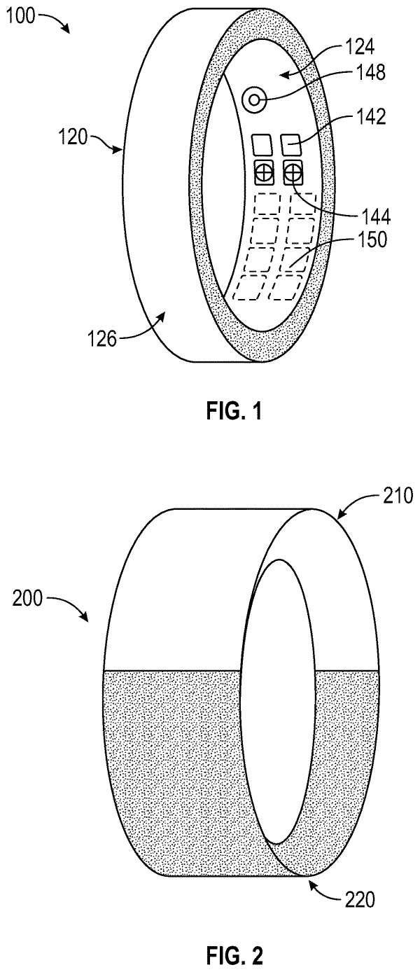

Referring to FIG. 1, an embodiment of a bioimpedance ring sensor 100 is illustrated. In the embodiment of FIG. 1, the bioimpedance ring sensor 100 includes a ring-shaped body 120, a plurality of electrodes 140, and a controller.

As will be discussed herein, the disclosed bioimpedance ring sensor(s) allows for bioimpedance sensing via an unobtrusive wearable form, for example, a ring sensor that can be worn at a convenient location such as the fingers of the wearer. Moreover, and as will also be disclosed herein, the disclosed bioimpedance ring sensor allows for the capture of a bioimpedance signal via electrodes of various sizes and numbers configured to be deployed in a ring sensor as disclosed herein, for example, in close, firm contact with the skin. As also discussed herein, the disclosed bioimpedance ring sensor addresses various shortcomings of previous systems.

As will also be discussed herein, the disclosed bioimpedance ring sensor(s) inject high-frequency low-amplitude alternating current into an individual's tissue to measure voltage potential changes due to body composition, blood flow via the arteries, and other physiological parameters such as arterial compliance and/or stiffness. Specifically, the disclosed bioimpedance ring sensor(s) has advantages, over other modalities, due to deep tissue penetration, reduced power consumption, and the ability to use close proximity electrodes.

As will be discussed herein, the disclosed bioimpedance ring sensor(s) enable placement of electrodes in closer proximity to arteries, for example, placement directly over arterial sites, yielding a bioimpedance signal that is representative of elastic arterial wall expansion, not intending to be bound by theory, due to arriving pulse waves and accompanying harmonic reflections.

Referring to FIG. 1, the ring-shaped body 120 may be characterized as having a central axis, an inner annular surface 124, and an outer annular surface 126. In operation, for example, when worn by the wearer, the inner annular surface may be in contact with the skin of the finger of the human subject, and the radially outer annular surface may face radially outward or away from the finger of the human subject with respect to the central axis.

The ring-shaped body 120 may, in various embodiments, have any suitable size as necessary to support a desired number and arrangement of electrodes, as disclosed herein. In various embodiments, the ring-shaped body 120 may have differing widths. For example, as will be disclosed herein, a ring-shaped body 120 having a relatively larger width can be capable of supporting a greater number of electrodes and/or relatively larger electrodes.

In various embodiments, the ring-shaped body 120 may be provided in various sizes (e.g., ring-sizes), for example, as suitable for various users requiring different sizes, and as necessary to provide the utility disclosed herein and fidelity of the bioimpedance ring sensor. For example, the bioimpedance ring sensor 100 may be provided in sizes and fitments correlating to conventional rings (i.e., jewelry).

In various embodiments, the ring-shaped body 120 can be rigid, flexible, or may comprise two or more portions that vary in flexibility and/or rigidity. For example, FIG. 2 illustrates an embodiment of a bioimpedance ring sensor 200 including a ring-shaped body 120 having both a rigid portion 210 and a flexible portion 220. In some embodiments, and not intending to be bound by theory, the use of a ring-shaped body 120 that is flexible and/or partially flexible can improve the unobtrusiveness of the bioimpedance ring sensor and the overall user-experience of the wearer and, as such, improving the probability that the bioimpedance ring sensor will be used long-term and across a variety of settings (e.g., ambulatory and nocturnal settings).

In some embodiments, the ring-shaped body 120 may be characterized as elastic. Not intending to be bound by theory, and as will be further discussed herein, an elastic ring-shaped body 120 may improve the reliability and/or sufficiency of contact between the electrodes 140 and the wearer's skin, thereby improving the accuracy of the bioimpedance signal that is detected by the bioimpedance ring sensor and the duration over which the bioimpedance signal is received, for example, by decreasing interruptions in the signal and/or decreasing background “noise” associated with the signal.

In various embodiments, the ring-shaped body 120 may be formed of any suitable material or combination of materials. Examples of suitable materials include metals/rigid materials such as silver, gold, copper, tungsten, titanium, stainless steel, ceramic, glass, flexible materials such as plastics, resins, silicone, and elastomers such as rubber. In some embodiments, the ring-shaped body 120 may include materials conventionally associated with rings (e.g., jewelry), for example, such that the bioimpedance ring sensor 100 appears similar to a conventional ring (jewelry). Additionally, the ring-shaped body 120 may include or be configured to receive ornamentation, for example, precious stones, again, such that the bioimpedance ring sensor 100 appears similar to a conventional ring (jewelry).

The electrodes 140 may be configured to measure the bioimpedance of the body of the subject being monitored, that is, to measure of electrical impedance of the subject's body tissue and fluid content. More particularly, the electrodes 140 may be configured to apply or “inject” a low-amplitude, high-frequency alternating current into the body of the subject being monitors and to sense or measure the resulting voltage potential. For example, in various embodiments, the alternating current may have an amplitude of from about 10 ρA to about 10 mA. Additionally or alternatively, in various embodiments, the alternating current may have a frequency from about 10 Hz to about 1 MHz, additionally or alternatively, from about 1 kHz to about 100 kHz.

In some embodiments, the electrodes 140 comprises both two injection electrodes 142 and two sensing electrodes 144. The injection electrode(s) 142 may be configured to apply or inject the low-amplitude, high-frequency alternating current into the body of the wearer and the sensing electrode(s) 144 may be configured to sense or measure the voltage potential form the body of the wearer. For example, the electrodes 140 may generally configured to provide contact with the skin of the wearer so as to facilitate ionic transfer between the skin of the electrode.

For examples, in some embodiments, one or more of the electrodes may comprise a surface configured for contact with the skin, referred to herein as a contact surface, that exhibits a curvature substantially conforming to the curvature of a wearer's finger. For example, in various embodiments the contact surface of one or more of the electrodes 140 may have a curvature corresponding to a radius of from about 6 mm to about 15 mm, for example, a curvature corresponding to a radius of about 6 mm, alternatively, about 7 mm, alternatively, about 8 mm, alternatively, about 9 mm, alternatively, about 10 mm, alternatively, about 11 mm, alternatively, about 12 mm, alternatively, about 13 mm, alternatively, about 14 mm, alternatively, about 15 mm. Not intending to be bound by theory, an electrode having a curved contact surface may exhibit improved conformity to the skin and/or increased surface area in contact with the skin.

Additionally or alternatively, in some embodiments one or more of the electrodes may be characterized as having a contact surface having an area of from about 1 mm² to about 50 mm², additionally or alternatively, an area of at least about 1 mm², 2 mm², 3 mm², 4 mm², 5 mm², 6 mm², 7 mm², 8 mm², 9 mm², 10 mm², 11 mm², 12 mm², 13 mm², 14 mm², 15 mm², 16 mm², 17 mm², 18 mm², 19 mm², or 20 mm² and/or less than about 50 mm², 40 mm², 30 mm², 25 mm², 24 mm², 23 mm², 22 mm², 21 mm², 20 mm², 19 mm², 18 mm², 17 mm², 16 mm², 15 mm², 14 mm², 13 mm², 12 mm², 11 mm², 10 mm², 9 mm², 8 mm², 7 mm², 6 mm², 5 mm², 4 mm², 3 mm², or 2 mm². In various embodiments, two or more of the electrodes may have the same or substantially the same size and/or exhibit the same or substantially the same curvature; additionally or alternatively, two or more of the electrodes may have a different size and/or exhibit a different curvature.

In some embodiments the electrode may be characterized as exhibiting adhesivity with respect to the wearer's skin. For example, in some embodiments at least a portion of the contact surface of one or more of the electrodes 140 may be formed from a material that is adhesive to skin and/or may be coated with an adhesive composition. The electrodes 140 may also be made of a suitably ionically-conductive material, for example, so as to facilitate ionic transfer between the skin and the electrode. In various embodiments, one or more of the electrodes 140 (for example, the injection electrode(s) 142 and/or the sensing electrode(s) 144) may comprise materials characterized as rigid, pliable, or flexible. For examples, in various embodiments the electrodes may comprise a metal (such as silver, gold, or alloys including silver or gold) a polymeric material (such as conductive silicone), a resin, carbon nanomaterials (such as carbon nanotubes), an highly conformal materials to the skin such as graphene, and combinations thereof. For example, in some embodiments one or more of the electrodes comprises a first material doped with another material, such as a polymeric material doped with an ionically conductive material. As an example, one or more of the electrodes may comprise a metal or carbon nanotube-doped silicone. Not intending to be bound by theory, an electrode formed of a pliable or flexible material such as silicone doped with an ionically conductive material such as a carbon nanomaterial may provide both improved conformability and electrode-to-skin contact and good ionic conductivity.

In addition, one or more of the electrodes may further comprise a biasing member, such as one or more spring, generally configured to improve the consistency of contact between the electrode and the skin of the wearer at all times.

Generally, the electrodes, for example, the injection electrode(s) 142 and sensing electrodes 144, are generally disposed on, in, and/or proximate the inner annular surface 124 of the ring-shaped body 120. In various embodiments, the electrodes 140 may be present in any suitable number. For example, in the embodiment of FIG. 1, the bioimpedance ring sensor 100 comprises four electrodes 140, particularly, two injection electrodes 142 and two sensing electrodes 144. For example, in some embodiments the electrodes 140 may be configured to utilize “Four Point Sensing.” The term “Four-Point Sensing” refers to a configuration of electrodes comprising at least four electrodes, including a positive injection terminal (I+), negative injection terminal (I−), positive voltage terminal (V+), and negative voltage terminal (V−). Not intending to be bound by theory, the injection of current at a high frequency, for example, a frequency from about 1 kHz to about 100 kHz, may be effective to enable the current to pass through the cell membranes, extracellular, and intracellular fluids of the body, thereby capturing comprehensive information about tissue and fluid content. Also not intending to be bound by theory, Four-point Sensing in conjunction with the injection of a high frequency current may help to ensure the injected current penetrates deep down into the arteries such that changes in blood volume and/or along static body information can be captured for physiological analysis. Additionally, Four-point Sensing in conjunction with the injection of a high frequency current may provide improved bioimpedance signals and avoid taking into account electrode-skin impedance.

Generally, the two or more of the injection electrodes 142 can be configured, for example, via the operation of the controller, to inject the same or substantially the same frequency. Not intending to be bound by theory, the provision of the same frequency injection by different injection electrodes 142 may yield a relatively higher coverage of the sensing area, for example, to mitigate the effect of bones and muscles that can block the injected current.

Additionally or alternatively, in some embodiments, the two or more of the injection electrodes 142 can be configured, for example, via the operation of the controller, to inject different frequencies of electric current. Not intending to be bound by theory, the provision of different frequencies by different injection electrodes 142 may isolate different sensing areas. For example, when different frequencies are injected, it is possible to determine the location of the bioimpedance signal injected by particular injection electrodes 142, which can be used to regenerate a bioimpedance signal related to flow. For example, a frequency domain analysis may be used to separate various bioimpedance signals captured at different injection frequencies. Source separation algorithms can be used to localize various sources of blood flow, extract the mutual blood flow information from these bioimpedance signals, and help to augment sensing fidelity.

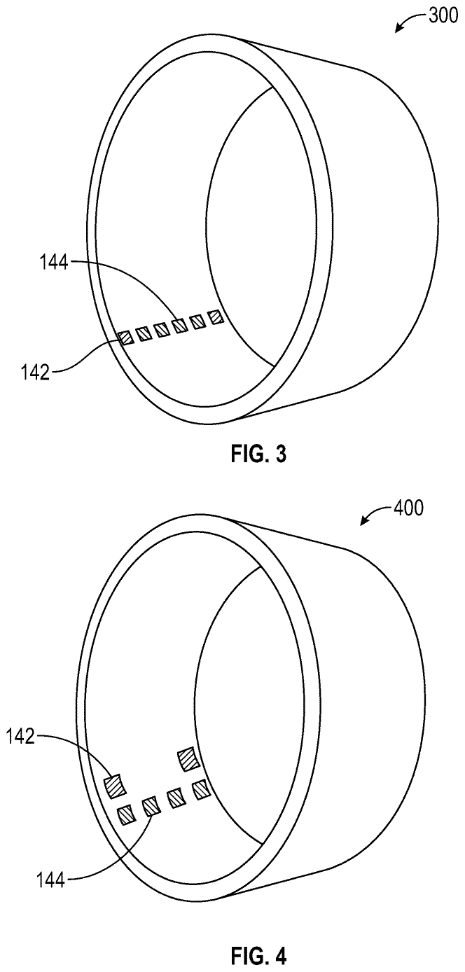

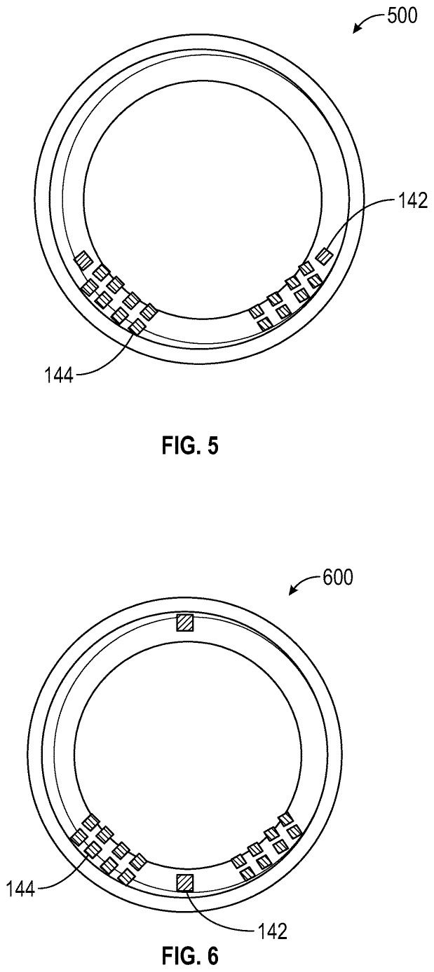

The injection electrodes 142 may be disposed on, in or proximate the inner annular surface 124 in any suitable arrangement. For example, as illustrated in the embodiment of FIGS. 3 and 4, a bioimpedance ring sensor 300, 400 may comprise two or more injection electrodes 142 disposed relatively close to each other so as to provide sensing in a localized area. Additionally or alternatively, as illustrated in the embodiment of FIG. 5, a bioimpedance ring sensor 500 may comprise two or more injection electrodes 142 disposed at a distance such that, for example, the two or more injection electrodes 142 are separated by some other component such as a sensing electrode 144, so as to provide a semi-localized area of sensing. Additionally or alternatively, as illustrated in the embodiment of FIG. 6, a bioimpedance ring sensor 600 may comprise two or more injection electrodes 142 disposed at opposite or substantially opposite sides of the ring-shaped body 120, for example, to provide a complete area of sensing. For example and not intending to be bound by theory, the disposition of two or more injection electrodes 142 at opposite or substantially opposite sides of the ring-shaped body 120 may allow for localization of the bioimpedance measurements, which may indicate parameters such as blood flow, muscle contractions, and tissue composition. Also for example and not intending to be bound by theory, the disposition of two or more injection electrodes 142 at opposite or substantially opposite sides of the ring-shaped body 120 may provide for relatively wider coverage so as to enable monitoring of overall changes in the finger circumference.

Generally, the two or more of the sensing electrodes 144 can be configured to sense the voltage potential from the body, for example, resulting from the injection of the voltage via the injection electrodes 142. Additionally, and as will be disclosed herein, the voltage potential sensed via the sensing electrodes 144 may be used to determine, via the controller, one or more parameters about the body.

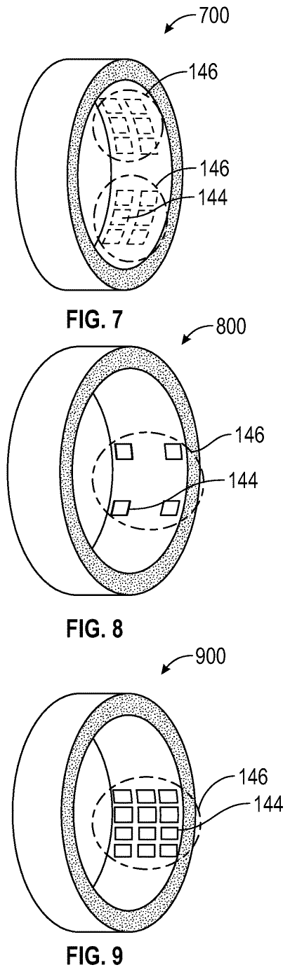

In some embodiments, the sensing electrodes 144 may be disposed in one or more sensing areas 146 on, in, or proximate the inner annular surface 124. For example, FIG. 7 illustrates a bioimpedance ring sensor 700 comprising the sensing electrodes 144 disposed in different sensing areas 146, which may have different combinations of electrodes used in current injection and voltage sensing.

In some embodiments, different sensing areas 146 may be employed to ascertain different measurements, for example, to obtain data indicative of different physiological parameters such as blood flow in the finger arteries. Additionally, for example, different sensing areas 146 can be used to provide optimum sensing of the area being monitored so as to provide the highest sensitivity with respect to the underlying blood flow of the proximate arteries. In various embodiments, the combined information from multiple sensing areas can be used to provide additional redundancy and/or to improve overall signal-to-noise ratio of the system.

Referring to FIGS. 8 and 9, in various embodiments, a bioimpedance ring sensor 800, 900 may include two or more sensing areas 146 including different numbers of sensing electrodes 144. In various embodiments, a sensing area 146 may include a single sensing electrode 144 or multiple sensing electrodes 144 to capture the bioimpedance signal at multiple locations, for example, 2, 3, 4, 5, 6, 7, 8, 9, 10, 11, 12, 13, 14, 15, 16, 17, 18, 19, 20, or more sensing electrodes 144. For example, a sensing area 146 may include multiple sensing electrodes 144, the outputs of which can be combined to manipulate the location of sensing of the current from the tissue and to capture bioimpedance data, as may be indicative of various physiological parameters such as blood flow, with the highest sensitivity. In some embodiments, the use of multiple sensing electrodes 144 can allow the capture of data indicative of a time difference in blood flow between two sensing locations which can be used to provide an estimate of blood flow velocity and pulse wave velocity. Additionally, in some embodiments multiple sensing electrodes 144 in array, for example, in a sensing area 146, can be activated spontaneously to perform a sweep of measurement one by one at each electrode to extract data indicative of finger tomography and/or to capture data indicative of muscle activations and/or artery localization.

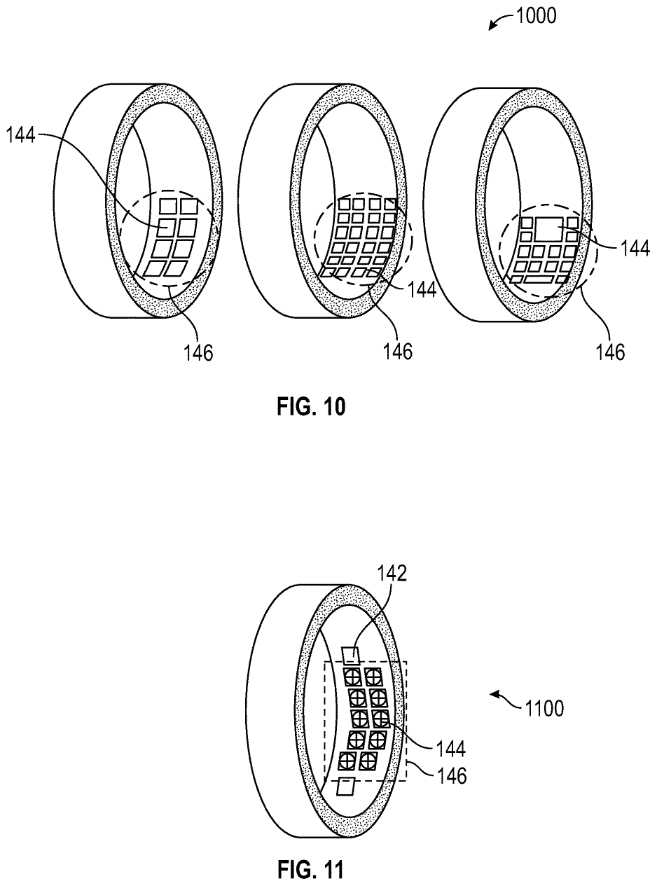

In some embodiments, a sensing area 146 may include various sizes of sensing electrodes. As illustrated in the embodiment of FIG. 10, the sensing area 146 may include multiple sizes of sensing electrodes, for example, relatively small-sized sensing electrodes 144, relatively large-sized sensing electrodes 144, or any suitable combination of differently-sized sensing electrodes 144. For instance, a sensing area can utilize a combination of small and large electrodes. Not intending to be bound by theory, multiple sensing electrodes 144 may be combined to achieve a higher surface contact area for better signal quality, to yield improved control on the data gather by the sensing area by selecting various combinations of sensing electrodes 144 which can be combined together for sensing purposes and/or which can be used in combination with particular injection electrodes 142. Also not intending to be bound by theory, for same bioimpedance ring sensor 100 of a given size, the number of electrodes that can be fit into the bioimpedance ring sensor 100 increases with the usage of relatively small-sized electrodes.

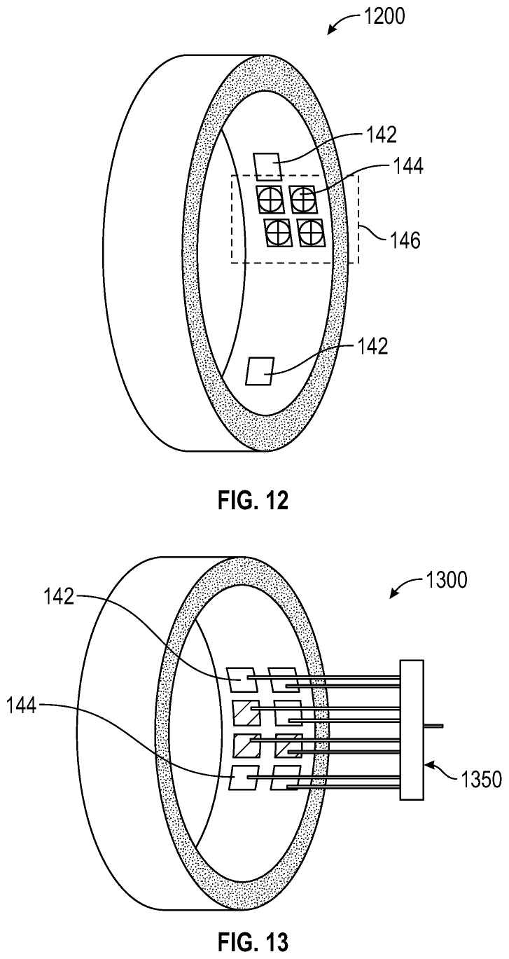

Referring to the embodiment of FIG. 11, a bioimpedance ring sensor 1100 may comprise different sensing electrodes 144 relative to a single injection electrode 142 configuration, as previously disclosed. For example, as illustrated in the embodiment of FIG. 11, multiple sensing electrodes 144 can be distributed between two or more injection electrodes 142. Also for example, as illustrated in the embodiment of FIG. 12, a bioimpedance ring sensor 1200 may comprise sensing electrodes 144 that can be placed relative closer to one of the injection electrodes 142 than another injection electrode 142. Not intending to be bound by theory, the use of multiple sensing electrodes 144 at different locations can allow the measurement of pulse transit time, pulse wave velocity, and arterial stiffness. In addition, and again not intending to be bound by theory, the use of multiple sensing electrodes 144 at different locations may also provide multiple observations of the blood flow, may be used to increase the redundancy and signal-to-noise ratio, and/or may provide a higher resolution of various tissue compositions (e.g., fat, muscle, artery).

Using wider rings allow the opportunity to place additional arrays of electrodes along the artery. In addition to the advantages of using multiple arrays of electrodes, a higher width results in a higher separation between the most left and most right electrodes. Therefore, the bioimpedance signal sensitivity to the blood flow will increase with this high separation due to the increase in the active sensing area.

In various embodiments, the electrodes, for example, the injection electrodes 142 and sensing electrodes 144, may be fully integrated into the ring-shaped body 120 such that the various electrodes are have a fixed relationship to each other and, also, such that the electrodes remain substantially fixed with respect to the body of the wearer, such as in the form of an “electric-tattoo.” Not intending to be bound by theory, the fixed relationship/orientation of the electrodes may be effective to ensure that the electrode-skin connection exhibits little or no movement over the time that the bioimpedance ring sensor is worn, such as might result from finger movements, and thereby improves the accuracy collected data, which may be indicative of blood flow and other hemodynamics measurements.

In some embodiments, for example, as illustrated schematically with respect to FIG. 13, a bioimpedance ring sensor 1100 may comprise one or more multiplexers 1350 can be included to enable that smart selection of the electrodes used for sensing and injection. For example, the operation of the multiplexer can be controlled within the ring or with an external device. In one embodiment, the multiplexers can be used to sweep the current injection and/or sensing electrode locations over all available electrodes to find the optimum area of sensing that gives highest sensitivity to blood flow.

In some embodiments, for example, as illustrated with respect to FIG. 1, one or more electrodes may be configured as a ground electrode 148, which may be effective to increase signal quality by having a common potential point. Not intending to be bound by theory, the utilization of one or more ground electrodes 148 may provide a common reference potential point with respect to the wearer's skin and, thereby, increase the common-mode rejection ratio (CMMR), resulting in a higher quality bioimpedance signal.

Generally, the controller may be configured to control the operation of the various components (e.g., the injection electrode(s) 142, sensing electrode(s) 144, and/or any other component) and/or receive signals from one or more of these components so as to determine bioimpedance associated with the body of the wearer, as disclosed herein.

Generally, the controller may comprise a processor and memory, wherein the processor is configured to execute machine-readable instructions stored on the memory to provide the processor (or more broadly the controller) with the functionality as disclosed herein. Thus, the memory may comprise a non-transitory machine-readable medium. In various embodiments, the processor may comprise any suitable configuration, for example, one or more microprocessors. The controller may also comprise one or more components or modules as necessary for the functionalities disclosed herein. For example, the controller may also comprise communication interface. Generally, the controller may be disposed on the inner annular surface 124 or within the ring-shaped body 120 and may be provided with suitably coupled to the injection electrodes 142 and sensing electrodes 144.

The bioimpedance ring sensor may also comprise a battery (e.g., a rechargeable battery, such as a lithium ion battery), which may provide power to the various components of the bioimpedance ring sensor. The battery may be disposed within the controller or otherwise within a portion of the bioimpedance ring sensor.

In some embodiments, the controller may be configured to receive one or more inputs, for example, via a user interface and to control the various components based upon the inputs from the user interface. In various embodiments, the user interface is in signal communication with the controller, for example, via a wireless connection such as near field communication (NFC), Wi-Fi, or Bluetooth. More specifically, the user interface allows a user to control and monitor the bioimpedance ring sensor such as via a wireless connection. The user interface may be designed to be user-friendly and intuitive, allowing a user to control and monitor the wearable therapy device using a wireless connection. The user interface can be accessed using a mobile device, tablet, or computer. For example, the user interface may comprise a graphical user interface (GUI) that is displayed on a mobile device, tablet, or computer.

The user interface allows the user to provide an indication of which physiological parameters the user wishes to monitor and the controller may cause the injection electrodes 142 to inject a current and the sensing electrodes 144 to sensing the resultant current effective to monitor the selected parameters, for example, by controlling which electrodes and/or other components are operated and at what location, frequency, intensity, voltage, and/or duration.

The user interface can be customized to meet the needs of different users or medical professionals. For example, the user interface may include different languages or font sizes to accommodate users with different backgrounds or visual impairments. The user interface may also include different modes or profiles for different types of monitoring or users. The user can download an application or access a web portal to connect with the medical device. The user interface may also include security features, such as passwords or biometric authentication, to ensure that only authorized users can access the device.

Additionally, the user interface may allow the user to monitor the bioimpedance ring sensor, adjust settings, start and/or stop monitoring, and view real-time data from the bioimpedance ring sensor. The user interface may also provide alerts or notifications when the wearable user device requires attention (e.g., a low battery alarm) or when certain conditions are met (e.g., when data indicates a health event).

The controller may be configured to direct an electric current to at least one of the injection electrodes 142 and to detect voltage potential via at least one of the sensing electrodes 144. Also, the controller may be configured to determine the bioimpedance associated with the body of the wearer and/or various physiological parameters derived therefrom, for example, based upon the electric current and the voltage potential. Generally, bioimpedance may be determined using Ohm's Law (V=I*Z), where bioimpedance is calculated by dividing the measured voltage signal by the known current injected. The determined bioimpedance may then be used (e.g., by the controller or another controller or computing system communicatively coupled to the controller) to determine one or more attributes or parameters of the human subject as disclosed herein.

In various embodiments, the bioimpedance ring sensor may include one or more additional components, for example, which may enable the bioimpedance ring sensor to be utilized in determining various additional parameters.

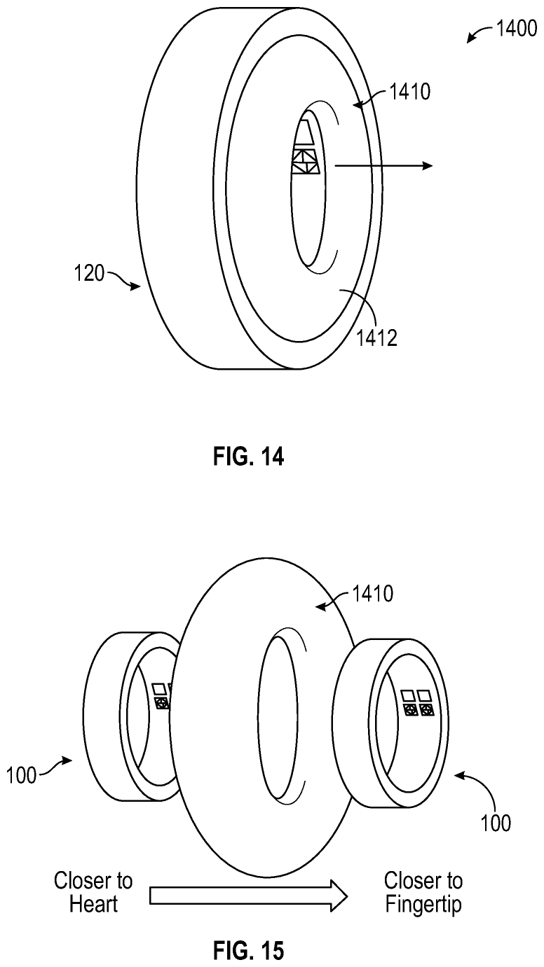

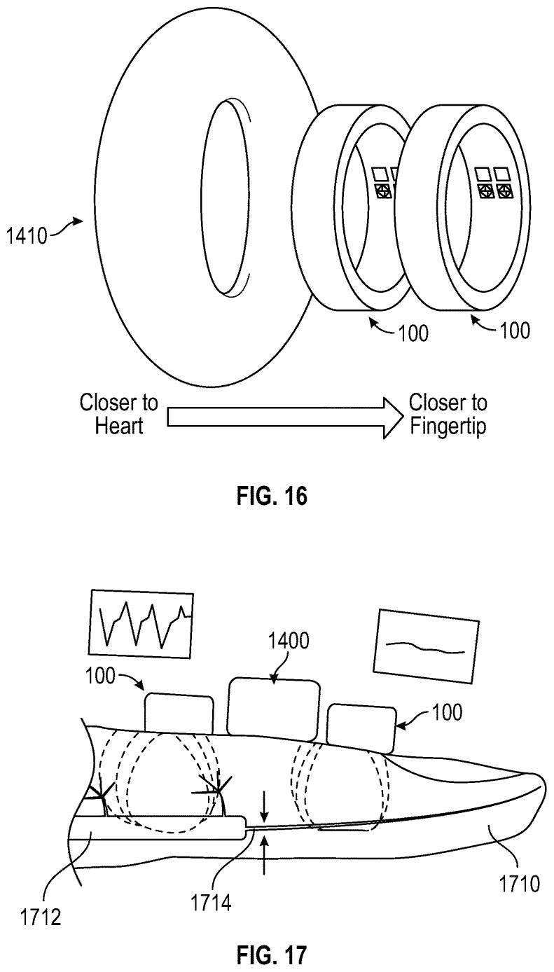

For example, referring to the embodiment of FIG. 14, an embodiment of a the bioimpedance ring sensor 1400 may include an inflatable cuff 1410 that is coupled to the ring-shaped body 120. Alternatively, referring to FIGS. 15 and 16, in some embodiments an inflatable cuff 1410 may be provided as a separate component, for example, not integrated into the bioimpedance ring sensor 100. For example, in the embodiments of FIGS. 15 and 16, the inflatable cuff 1410 is illustrated as a separate component that can be used with a bioimpedance ring sensor such as the bioimpedance ring sensor 100 of FIG. 1.

The inflatable cuff 1410 may comprise an annularly (or ring)-shaped cuff body 1412 circumferentially positioned about a cuff axis and an inflation assembly coupled to the cuff body 1412 that is configured to selectively increase an internal volume of the cuff body. The cuff body may be positioned along the radially inner annular surface of the ring-shaped body or may be separate from the ring-shaped body. During operations, and as illustrated in FIG. 17, the cuff body 1412 may be placed about the finger 1710 of the human subject and the inflation assembly may be selectively actuated (e.g., by the controller) to selectively inflate and constrict fluid flow (e.g., blood flow) through the human subject's finger, for example, to form an artery occlusion 1714 at a digital artery 1712, as the controller detects bioimpedance via the plurality of electrodes as previously described. Not intending to be bound by theory, use of the inflatable cuff 1410 may allow additional useful parameters of the human subject to be detected an analyzed, such as, for instance, blood pressure.

The inflatable cuff 1410 may also comprise an inflation assembly that is configured to selectively adjust an internal volume within the cuff body 1412. The inflation assembly may be communicatively coupled to the controller, for example, such that the inflation assembly may control the inflation of the cuff body 1412. The inflation intensity of the cuff body 1412 can be adjusted to manipulate the blood flow at the finger arteries and veins and measure various parameters including the blood volume changes in presence small, medium or normal blood flow. This information can be used to derive various hemodynamic parameters including vascular resistance or vascular compliance or arterial stiffness. The cuff body 1412 can have an inner pressure sensor to sense the blood pressure applied to the arterial walls. This information can also be used to calibrate the hemodynamic parameter estimation using bioimpedance.

Additionally or alternatively, referring again to the embodiment of FIG. 1, the bioimpedance ring sensor 100 may include one or more additional sensors 150 or detectors, for example, that may be coupled to the ring-shaped body 120 or, in an alternative embodiment to another component such as the cuff body (if present). In some embodiments, the additional sensors or detectors may comprise one or more temperature detectors (e.g., temperature sensors, such as thermocouples, thermistors, etc.), accelerometers, gyros, or combinations thereof. The controller (or another computing system communicatively coupled to the controller) may utilize the output signals from the additional sensors or detectors to further enhance the detection or measurement of the one or more attributes or parameters of the human subject (or a system thereof such as the circulatory system) as previously described.

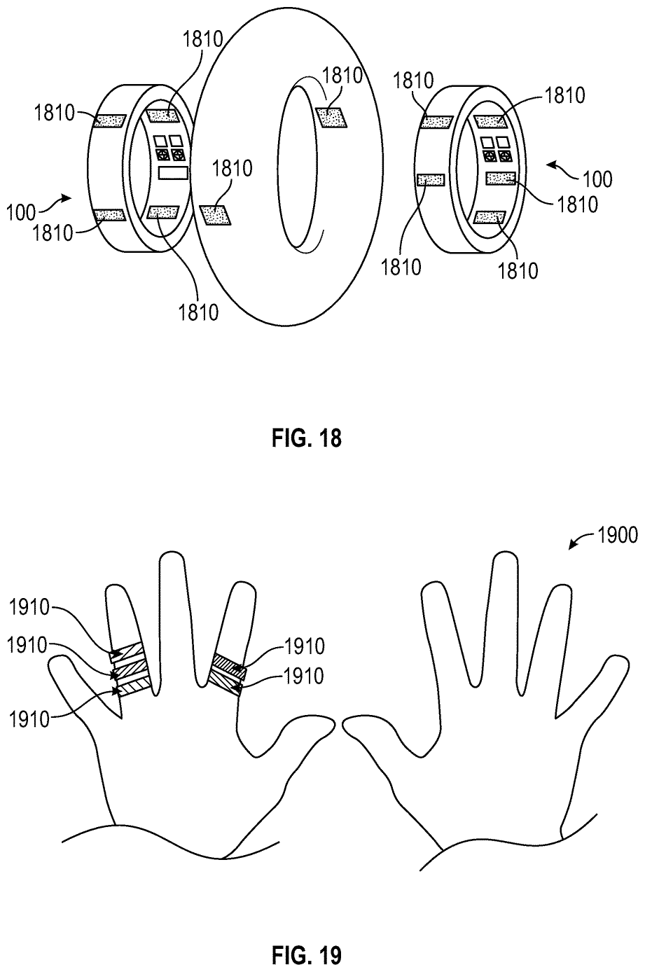

Referring to FIG. 18, in various embodiments, temperature detectors 1810 can be placed at the outer side of the bioimpedance ring sensor 100 and/or on or proximate the inner annular surface 124 of the bioimpedance ring sensor 100. For example, and again not intending to be bound by theory, the temperature detectors 1810 may provide both contextual information (for example, external temperature) and physiological information (for example, skin or body temperature changes due to blood flow or fever). In various embodiments, the data obtained from the temperature detectors may be used to calibrate blood flow measurements under different contextual settings, to calibrate and normalize blood flow measurements under different physiological health status(es), such as changes in the blood pressure due to stress and disease state that also can alter the skin and core body temperature. In some embodiments, multiple temperature detectors can be used together to provide data to enable localizing the finger arteries and can guide the activation of bioimpedance electrodes with specific area of sensing.

In some embodiments, the bioimpedance ring sensor 100 can also use accelerometers and/or gyroscopes to detect information indicative of the hand position. In some embodiments, the information indicative of hand position can be used to calibrate the biometric information and/or can also be used for signal processing, such as for the removal of motion artifacts that degrade signal quality. Also, in some embodiments, to improve longevity with respect to power consumption, the controller can be configured to activate at different times (for example, in a number of intermittent cycles) to conserve power (e.g., duty cycling). For example, data from an accelerometer may be used to cause the bioimpedance ring sensor to undergo a “wake-up” process. Additionally, in some embodiments, accelerometer data can be used to provide additional biometric and contextual information. In some embodiments, the bioimpedance ring sensor 100 can also comprise an optical sensor, for examples, a photoplethysmography sensor.

The ring measures bioimpedance to construct physiological/biometric information, including hemodynamic and cardiovascular parameters (e.g., blood pressure, blood volume changes, artery compliances), electro-dermal activity, muscle contractions, body composition changes. A frequency sweep on the ring can be performed to capture the tissue composition and to assess hydration.

In some embodiments, a bioimpedance ring sensor, for example, one or more of bioimpedance ring sensors 100, 200, 300, 400, 500, 600, 700, 800, 900, 1100, 1200, 1300, and/or 1400 as disclosed with respect to one or more of the Figures disclosed herein may be utilized as a part of a bioimpedance system.

In some embodiments, the bioimpedance system may generally include one or more bioimpedance ring sensors. For example, in various embodiments the bioimpedance system may comprise one or more (e.g., a plurality of) bioimpedance ring sensors for detecting bioimpedance of the finger or fingers of a human subject as previously described. For instance, in some embodiments, each of the one or more bioimpedance ring sensors may be placed about one finger or multiple fingers of the human subject. In some embodiments, at least two of the bioimpedance ring sensors may be physically coupled to one another, for example, such that the at least two bioimpedance ring sensors are coaxially aligned.

In some embodiments, the at least two bioimpedance ring sensors may have engaged connectors (e.g., electrical connectors, fiberoptic connectors, etc.) that facilitate communication between the at least two bioimpedance ring sensors (or more particularly between the controllers of the two bioimpedance ring sensors). In some embodiments, a plurality of bioimpedance ring sensors (or more particularly the controllers of the plurality of bioimpedance ring sensors) may communicate wirelessly with one another, whether the plurality of ring-shaped bodies are physically engaged or separate from one another.

For example, in some embodiments, a first bioimpedance ring sensor may comprise a first connector positioned on the first end of the ring-shaped body and a second bioimpedance ring sensor may comprise a second connector positioned on the second end of the second ring-shaped body. The first connector may be configured to engage the second connector when the first end of the ring-shaped body is engaged with the second end of the second ring-shaped body such that the central axis and the second central axis are coaxially aligned, thereby enabling the controllers of the first and second bioimpedance ring sensors to communicate with the second controller via the first connector and the second connector when the first connector and the second connector are engaged.

In various embodiments, the bioimpedance system may include at least one bioimpedance ring sensor used with another sensor (e.g., another bioimpedance ring sensor) in any suitable combination or arrangement.

For example, referring to the embodiment of FIG. 19, an embodiment of bioimpedance system 1900 comprising a plurality of bioimpedance ring sensors 1910 is illustrated. As illustrated, multiple bioimpedance ring sensors 1910 can be worn on the same finger. For example, and not intending to be bound by theory, multiple bioimpedance ring sensors 1910, when used together, on the same finger may provide various advantages. For example, the bioimpedance measurements from multiple bioimpedance ring sensors 1910 may provide bioimpedance (e.g., blood flow measurement) data at multiple locations, and the differences in bioimpedance between different sensing locations can be used to determine important hemodynamic parameters like pulse transit time (PTT) that has a strong correlation with complex cardiac biometric information such as blood pressure (BP). Also, for example, bioimpedance measurements from multiple bioimpedance ring sensors 1910 may provide a higher sensing area along an artery in the subject's finger to increase sensing signal quality. For example, a first bioimpedance ring sensor 1910 can have positive poles for injection and/or sensing, and a second bioimpedance ring sensor 1910 can have negative poles for injection and/or sensing; this arrangement may increase the coverage and it may also improve the bioimpedance signal quality.

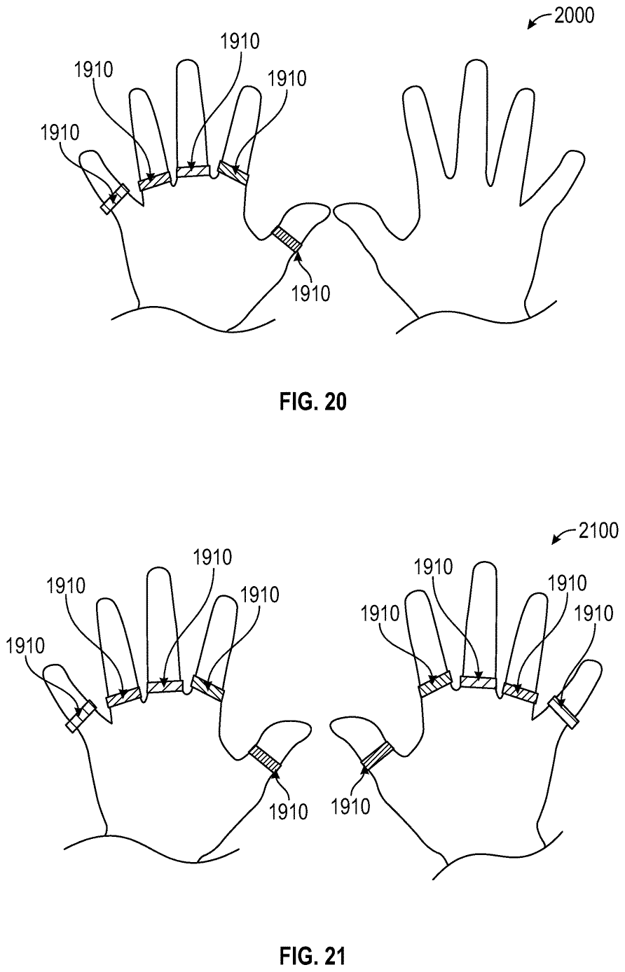

Also for example, referring to the embodiment of FIG. 20, another embodiment of bioimpedance system 2000 comprising a plurality of bioimpedance ring sensors 1910 is illustrated. As illustrated, multiple bioimpedance ring sensors 1910 can be on different fingers of the same hand. For example, and not intending to be bound by theory, multiple bioimpedance ring sensors 1910, on different fingers of the same hand, can provide multiple readings of bioimpedance data, such as blood flow, at different parts of the circulation system over the hand and may both increase the fidelity of the blood flow and other physiological measurements when the information from multiple bioimpedance ring sensors 1910 is combined, also, provide additional hemodynamic information such as pulse wave velocity due to different arrival times of the blood pressure pulse wave at different fingers.

Also for example, referring to the embodiment of FIG. 21, another embodiment of bioimpedance system 2100 comprising a plurality of bioimpedance ring sensors 1910 is illustrated. As illustrated, multiple bioimpedance ring sensors 1910 can be placed on different hands. For example, and not intending to be bound by theory, multiple bioimpedance ring sensors 1910, on different hands, can provide additional information on the circulation system, due to the separate arterial paths supplying blood to different hands.

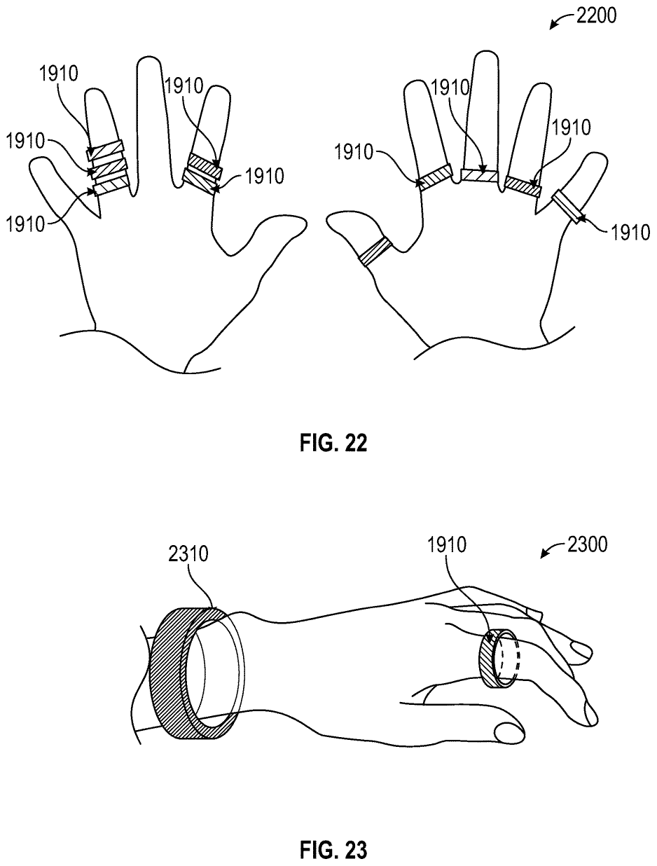

Also for example, referring to the embodiment of FIG. 22, another embodiment of bioimpedance system 2200 comprising a plurality of bioimpedance ring sensors 1910 is illustrated. As illustrated, multiple bioimpedance ring sensors 1910 can be placed on any combination of fingers on one or both hands to provide additional redundancy and measurement fidelity. For example, and not intending to be bound by theory, multiple rings worn on different fingers and/or different hands may capture simultaneous muscle contractions and blood flow. For example, muscle contractions can be used for hand gesture recognition that is useful for sign-language recognition. Blood flow detection at multiple fingers can assess the circulation within the digital arterial tree. The time delays and different signal morphologies from different fingers with individual digital arteries can provide hemodynamic information.

In some embodiments, a bioimpedance system may comprise at least one bioimpedance ring sensor, as disclosed herein, and one or more other components.

For example, referring to FIG. 23, an embodiment of a bioimpedance system 2300 comprising a bioimpedance ring sensor 1910 and a wrist sensor 2310 is illustrated, which may enable capture of a wider variety and quality of hemodynamic parameters. For example, the use of a bioimpedance ring sensor 1910 with a wrist sensor 2310 can provide information on blood flow such as the pulse wave velocity and pulse transit time, due to the simultaneous measurements of blood pressure pulse wave at a distal and proximal locations. In some embodiments, this information can be used to build cardiovascular estimation models, for example, blood pressure estimation and/or arterial stiffness estimation.

Also for example, referring to FIGS. 15 and 16, a bioimpedance system may comprise at least one bioimpedance ring sensor 100 and an inflatable cuff 1410. For example, as illustrated in FIG. 15, bioimpedance ring sensors 100 may be placed on each side of the inflatable cuff 1410, which may enable blood flow data to be collected before, during, and after the cuff inflation/deflation. This information may be used to determine blood volume changes and other blood hemodynamic properties and estimates of the arterial compliance. The combined information from bioimpedance ring sensors placed closer to heart (before the inflatable cuff) and further from the heart (after the cuff) can be used to track the blood volume changes during vasoconstriction and vasodilatation of the arteries. This information can improve measurements of the artery and its wall characteristics (e.g., artery compliance and stiffness), and can provide additional information including peripheral vascular resistance.

Also for example, as illustrated in FIG. 16, bioimpedance ring sensors 100 may be after the inflatable cuff, such that as the pressure of the inflatable cuff is changed, the bioimpedance can measured and the change in the blood flow can be determined at two locations. The combined information can be used to determine pulse transit time and pulse wave velocity information, in addition to artery wall and artery characteristics (e.g., artery compliance).

During operations, the bioimpedance ring sensor(s) may be placed on the finger of the human subject, and the controller may direct electrical current (e.g., derived from an on-board power source, such as a battery, capacitor, etc.) to one or more of the injection electrodes. The electrical current may travel from the injection electrodes through the finger of the human-subject, and a voltage may be detected via the controller via the sensing electrodes. The controller may then determine (e.g., calculate) the bioimpedance of the finger of the human subject based on the known electrical current provided to the first set of the electrodes and the voltage detected at the second set of the plurality of electrodes (e.g., via Ohms Law). The controller may also utilize the bioimpedance to determine one or more additional physiological parameters.

In some embodiments, the bioimpedance ring sensors and the bioimpedance systems disclosed herein can collect date and determine, based upon that data, blood pressure, arterial stiffness, respiration activity, heart rate and heart rate variability, tissue composition, fat ratio, hydration, muscle activities (EMI), and glucose levels. Additionally or alternatively, in some embodiments, the bioimpedance ring sensors and the bioimpedance systems disclosed herein can use the posture information to calibrate the biometric information. For example, the bioimpedance ring sensors and the bioimpedance systems disclosed herein can contain algorithms to compare the impact of posture on the ring measurements. For example, the bioimpedance ring sensors and the bioimpedance systems disclosed herein can compare standing posture (with known height and hand position) to a supine posture (e.g., during sleep). The bioimpedance ring sensors and the bioimpedance systems disclosed herein can use the ring data to detect additional biometric information such as peripheral arterial tone using the amplitude and phasic changes in the bioimpedance signal measured from the ring, along with the use of multiple bioimpedance signals measured at multiple locations simultaneously from either a single ring, multiple rings worn on the same, multiple fingers of the same hand, or different hands.

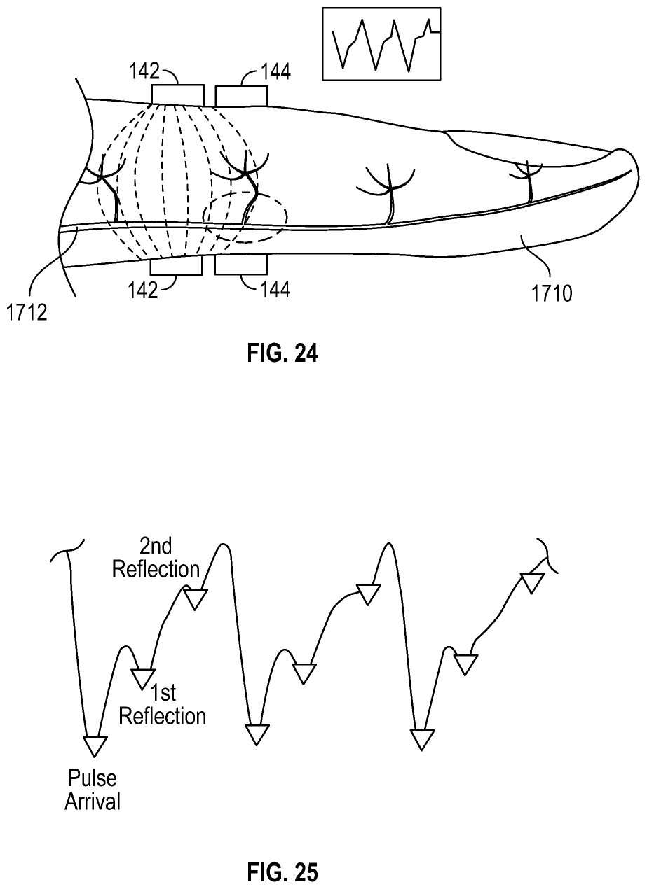

The bioimpedance ring sensors and the bioimpedance systems disclosed herein enable precision measurements of hemodynamic parameters ideally captured from a site where no complex arterial network is present. Particularly, the bioimpedance ring sensors and the bioimpedance systems disclosed herein are particularly configured to be employed on the finger, where the arterial network is relatively simple, for example, as illustrated in FIG. 24. Conversely, in other parts of the body, where a complex arterial network is present, it is more challenging to capture signals from a specific site of interest, where the measurements capture a complex supercomposition of hemodynamic parameters based on underlying blood flow occurring at multiple arterial sites at varying depths (e.g., radial and ulnar arteries at the wrist).

The bioimpedance ring sensors and the bioimpedance systems can non-invasively and unobtrusively measure continuous physiological biometrics from the user's fingers. The bioimpedance ring sensors and the bioimpedance systems are able to capture accurate information related to blood flow through the digital arteries using the deep tissue sensing enabled with bioimpedance modality with its unique design that helps to establish tight and/or suitable electrical contact with the wearer's skin, at all times, activation of electrodes, offering a convenient experience to the wearers, especially for long-term ambulatory and night time wear. Rings in general are most comfortable wearables, most users may choose to wear them continuously and they will very little discomfort if they are property sized.

Moreover, the bioimpedance ring sensors and the bioimpedance systems are compact and convenient to wear, and thereby provide seamless sensing of various physiological parameters. The bioimpedance ring sensors and the bioimpedance systems can accompany additional sensors and actuators, such as a miniaturized inflatable cuff that provides local occlusion of blood flow at the arteries and veins, temperature sensors to provide calibration readings and guidance for operation of the inflatable cuff and, accelerometer sensors to provide additional biometric and contextual information such as posture, hand motion, and activity. The measurements acquired with the bioimpedance ring sensors and the bioimpedance systems can be used to obtain complex physiological parameters such as blood pressure, heart rate, and respiration, along with additional useful biometric information. The disclosed technology utilizes bioimpedance sensing to provide personalized insight to an individual's cardiovascular health, body composition, and other physiological parameters.

### EXAMPLES

The following examples are provided to illustrate the present disclosure. The examples are not intended to limit the scope of the present disclosure and they should not be so interpreted.

### Example 1

When electrodes are directly placed over arterial sites, the captured high resolution bioimpedance signals are representative of elastic arterial wall expansion due to arriving pulse waves and accompanying harmonic reflections. Explicitly, the arrival of the blood pulse wave is indicated by the largest trough of the signal followed by reflections as shown in FIG. 25. Data collected from a group of five participants comprised of healthy normotensive individuals with an age ranged from 20 to 24, with three of the five being males and the other two females. The systolic BP ranges for all participants were from 91 to 171 mmHg and diastolic range from 51 to 95 mmHg. Tables I& II summarize the predicted systolic and diastolic errors in mean absolute error (MAE), standard deviation of the absolute error (STD), and root mean squared error (RMSE) for all participants and the average amongst them. Table Ill provides the percentage values that meet defined error thresholds in mmHg for all predicted data. These reported results from five participants helps to provide initial insight to the promising potential of utilizing ring-based bioimpedance systems for BP estimation.

TABLE I
Predicted Systolic BP Error (mmHg)

| Participant | MAE | STD | RMSE |
|---|---|---|---|
| S1 | 5.72 | 3.99 | 7.11 |
| S2 | 2.66 | 1.53 | 3.08 |
| S3 | 3.88 | 2.80 | 4.82 |
| S4 | 5.41 | 3.79 | 6.61 |
| S5 | 4.22 | 2.34 | 4.90 |
| Avg. | 4.38 | 2.89 | 5.30 |

TABLE II
Predicted Diastolic BP Error (mmHg)

| Participant | MAE | STD | RMSE |
|---|---|---|---|
| S1 | 4.25 | 2.64 | 5.06 |
| S2 | 2.29 | 1.64 | 2.83 |
| S3 | 2.42 | 1.59 | 2.91 |
| S4 | 5.89 | 3.80 | 7.03 |
| S5 | 3.31 | 2.43 | 4.13 |
| Avg. | 3.63 | 2.42 | 4.39 |

TABLE III
Threshold Error Percentage for Estimated Blood Pressures

| BP Phase | ≤5 mmHg | ≤10 mmHg | ≤15 mmHg | ≥15 mmHg |
|---|---|---|---|---|
| Systolic | 68% | 90% | 97% | 3% |
| Diastolic | 77% | 93% | 98% | 2% |

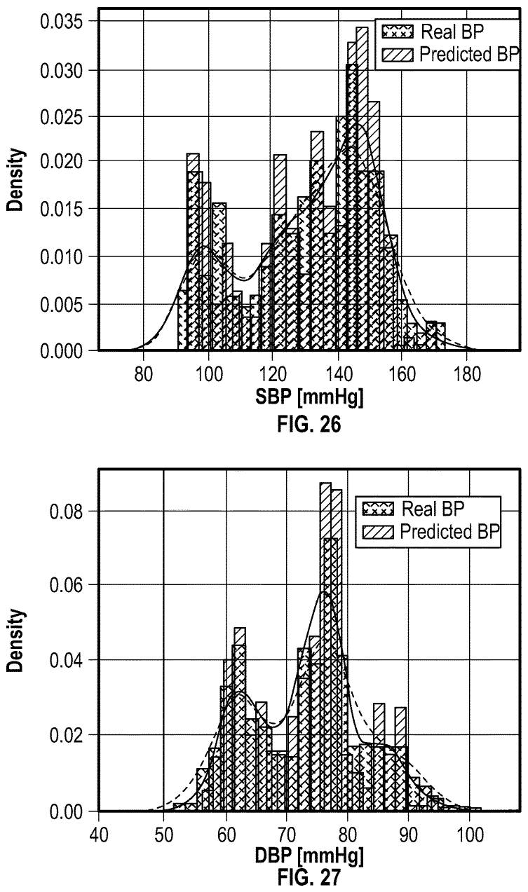

The statistical results amongst all five participants demonstrate promising potential with mean absolute errors, standard deviations, and root mean square errors all under 5 mmHg, except for the predicted systolic RMSE. Additionally, the system demonstrates 90-93% of predicted values having less than or equal to 10 mmHg of error. FIGS. 26 and 27 graphically display the histogram and density plots of the real BP and predicted BP, in which the ML models lacked in predicting values with increased presence within the dataset.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed, for example a range of the number of carbon atoms, molar ratios, temperatures, and the like, it is intended to disclose or claim individually each possible number that such a range could reasonably encompass, including any sub-ranges encompassed therein. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure, for example, to account for a reference that Applicants are unaware of at the time of the filing of the application.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

1. A bioimpedance ring sensor configured to be worn on a finger of a human subject, the bioimpedance ring sensor comprising:
a ring-shaped body having a central axis, a radially inner annular surface, and a radially outer annular surface;
a plurality of electrodes positioned on the radially inner annular surface, wherein the plurality of electrodes comprise a plurality of injection electrodes and a of plurality sensing electrodes; and
a controller coupled to the plurality of electrodes, wherein the controller is configured to direct an electric current to at least one of the plurality of injection electrodes, to detect a voltage potential via at least one of the plurality of sensing electrodes, and to determine bioimpedance associated with the human subject based upon the electric current and the voltage potential.

2. The bioimpedance ring sensor of claim 1, wherein the plurality of electrodes comprises at least four electrodes, wherein the plurality of injection electrodes comprises at least two injection electrodes, and wherein the plurality of sensing electrodes comprises at least two sensing electrodes.

3. The bioimpedance ring sensor of claim 1, wherein two or more of the plurality of sensing electrodes are disposed in a sensing region of the radially inner annular surface.

4. The bioimpedance ring sensor of claim 1, further comprising a multiplexer coupled to the plurality of electrodes, wherein the multiplexer is configured to select one or more of the plurality of injection electrodes and/or to select one or more of the plurality of sensing electrodes.

5. The bioimpedance ring sensor of claim 1, further comprising at least one ground electrode positioned on the radially inner annular surface.

6. The bioimpedance ring sensor of claim 1, further comprising an inflatable cuff comprising:
a cuff body circumferentially positioned about a cuff axis; and
an inflation assembly configured to selectively adjust an internal volume within the cuff body,
wherein the inflation assembly is communicatively coupled to the controller.

7. The bioimpedance ring sensor of claim 6, wherein the cuff body is positioned on, in, or proximate the radially inner annular surface of the ring-shaped body so that the cuff axis is coaxially aligned with the central axis.

8. The bioimpedance ring sensor of claim 1, further comprising a temperature detector coupled to the ring-shaped body, a motion sensor, or an optical sensor.

9. The bioimpedance ring sensor of claim 8, wherein the temperature detector is positioned on the radially inner annular surface.

10. The bioimpedance ring sensor of claim 8, wherein the temperature detector is positioned on the radially outer annular surface.

11. The bioimpedance ring sensor of claim 8, wherein the motion sensor comprises an accelerometer or a gyroscope.

12. The bioimpedance ring sensor of claim 8, wherein the optical sensor comprises a photoplethysmography sensor.

13. A system, comprising:
a first bioimpedance ring sensor configured to be worn on a finger of a human subject, the first bioimpedance ring sensor comprising:
a first ring-shaped body having a first central axis, a first radially inner annular surface, and a first radially outer annular surface;
a first plurality of electrodes positioned on the first radially inner annular surface, wherein the first plurality of electrodes comprise a first plurality of injection electrodes and a first plurality sensing electrodes; and
a first controller coupled to the first plurality of electrodes, wherein the first controller is configured to direct an electric current to at least one of the first plurality of injection electrodes, to detect a voltage potential via at least one of the first plurality of sensing electrodes, and to determine bioimpedance associated with the human subject based upon the electric current and the voltage potential.

14. The system of claim 13, wherein the first plurality of electrodes comprises at least four electrodes, wherein the first plurality of injection electrodes comprises at least two injection electrodes, and wherein the first plurality of sensing electrodes comprises at least two sensing electrodes.

15. The system of claim 13, wherein two or more of the first plurality of sensing electrodes are disposed in a first sensing region of the first radially inner annular surface.

16. The system of claim 13, further comprising a first multiplexer coupled to the first plurality of electrodes, wherein the first multiplexer is configured to select one or more of the first plurality of injection electrodes and/or to select one or more of the first plurality of sensing electrodes.

17. The system of claim 13, further comprising at least one ground electrode positioned on the first radially inner annular surface.

18. The system of claim 13, comprising:
a second bioimpedance ring sensor configured to be worn on a finger of a human subject, the second bioimpedance ring sensor comprising:
a second ring-shaped body having a second central axis, a second radially inner annular surface, and a second radially outer annular surface;
a second plurality of electrodes positioned on the second radially inner annular surface, wherein the second plurality of electrodes comprise a second plurality of injection electrodes and a second plurality sensing electrodes; and
a second controller coupled to the first plurality of electrodes, wherein the second controller is configured to direct an electric current to at least one of the second plurality of injection electrodes, to detect a voltage potential via at least one of the first plurality of sensing electrodes, and to determine bioimpedance associated with the human subject based upon the electric current and the voltage potential.

19. The system of claim 18, wherein the first controller and the second controller are configured to communicate with one another via a wireless signal.

20. The system of claim 18, wherein:
the first ring-shaped body has a first end and a second end opposite the first end along the first central axis;
the second ring-shaped body has a first end and a second end opposite the first end along the second central axis;
the first ring-shaped body includes a first connector positioned on the first end of the first ring-shaped body;
the second ring-shaped body includes a second connector positioned on the second end of the second ring-shaped body;
when the first connector is configured to engage the second connector when the first end of the first ring-shaped body is engaged with the second end of the second ring-shaped body such that the central axis and the second central axis are coaxially aligned; and
the first controller is configured to communicate with the second controller via the first connector and the second connector when the first connector and the second connector are engaged.

21. The system of claim 13, further comprising an inflatable cuff comprising:
a cuff body circumferentially positioned about a cuff axis; and
an inflation assembly configured to selectively adjust an internal volume within the cuff body,
wherein the inflation assembly is communicatively coupled to the first controller.

22. The system of claim 21, wherein the cuff body is positioned on, in, or proximate the first radially inner annular surface of the first ring-shaped body so that the cuff axis is coaxially aligned with the first central axis.

23. The system of claim 13, further comprising a temperature detector coupled to first the ring-shaped body.

24. The system of claim 23, wherein the temperature detector is positioned on the first radially inner annular surface.

25. The system of claim 23, wherein the temperature detector is positioned on the first radially outer annular surface.

26. A method of monitoring a physiological parameter of a human subject, the method comprising:
positioning a bioimpedance ring sensor on a finger of the human subject, the bioimpedance ring sensor comprising:
a ring-shaped body having a central axis, a radially inner annular surface, and a radially outer annular surface;
a plurality of electrodes positioned on the radially inner annular surface, wherein the plurality of electrodes comprise a plurality of injection electrodes and a plurality of sensing electrodes; and
a controller coupled to the plurality of electrodes;
directing an electric current to at least one of the plurality of injection electrodes;
detecting a voltage potential via at least one of the plurality of sensing electrodes; and
determining bioimpedance associated with the human subject based upon the electric current and the voltage potential.
